(19) United States
(12) Patent Application Publication
(10) Pub. No.: US 2010/0268180 A1
(43) Pub. Date: Oct. 21, 2010

(54) TEST APPARATUS

(75) Inventor: Edward Hartwell, York (GB)

Correspondence Address: KNOBBE MARTENS OLSON & BEAR LLP, 2040 MAIN STREET, FOURTEENTH FLOOR, IRVINE, CA, 92614, US

(73) Assignee: Smith & Neohew PLC, London (GB)

(21) Appl. No.: 12/808,648

(22) PCT Filed: Dec. 3, 2008
(86) PCT No.: PCT/GB08/51151

§ 371 Date: Jun. 16, 2010

(30) Foreign Application Priority Data
Dec. 20, 2007 (GB) 0724824.8

(51) Int. Cl. A61M 1/00; G01L 7/00

(52) U.S. Cl. 604/320; 604/319; 73/700

(57) ABSTRACT

A method and apparatus and an adapter device for testing the integrity of a dressing applied to a wound for the purpose of applying topical negative pressure therapy to that wound are described, the method comprising the steps of: applying a dressing to the wound so as to create a sealed cavity over the wound, the cavity having an aspiration conduit operably connected thereto for the purpose of creating a vacuum in the cavity; providing adapter device means to a liquid scalpel so as to create a substantially closed volume around a region in which a water jet exists in operation of the water scalpel; connecting a free end of the aspiration conduit to the closed volume formed by the adapter device means on the scalpel; and, using a vacuum created by a venturi effect of the liquid jet in the closed volume to create a vacuum in the dressing cavity.

The present invention relates to a method, apparatus and a device for testing the integrity of a dressing applied to a patient for the purpose of applying topical negative pressure (TNP) therapy.

For wounds to continue to heal it is sometimes necessary for the wound to be debrided. Debriding is the removal of dead tissue from the wound and removal is sometimes necessary because the dead tissue may harbour infection, may have become hardened, for example, and may form a barrier stopping or at least retarding new cell growth (indeed such dead tissue is often referred to as “a barrier to healing” in relevant literature).

Earlier techniques for debridement frequently made use of scalpels where the dead tissue was surgically removed, usually by a surgeon.

More recent techniques have made use of so-called water scalpels where a fine jet of water at very high pressure is used to effect tissue removal. Again the debridement is usually carried out by a surgeon in an operating theatre and is classed as a surgical procedure and carried out under sterile conditions. Once the wound has been debrided leaving a new clean wound surface there is usually bleeding to deal with and the wound needs to be dressed to help stem the bleeding and also to prevent infection.

For harder to heal wounds such as trauma wounds, for example, TNP therapy is often used and which requires a specific kind of dressing which covers the wound with an adherent sealing membrane so as to create a sealed cavity over the wound and which cavity is subsequently connected to a vacuum source so as to apply a reduced pressure (relative to ambient atmospheric pressure) in the wound cavity. The requirements of such TNP therapy dressings are well known to those persons skilled in the relevant art.

One of the problems of such TNP dressings is that they may leak, i.e. it is difficult to maintain a vacuum in the wound cavity since ambient air may be sucked into the cavity around the sealing membrane, for example. Ideally the TNP dressing should be applied in the operating theatre under sterile conditions to obviate infection. However, in order to test the dressing a vacuum needs to be applied to the cavity to determine if any leakage is sufficiently severe to warrant the dressing being replaced immediately. However, due to the need to apply a vacuum it is necessary for a vacuum device to be brought into the theatre and which vacuum device consequently needs to be sterile itself. This leads to considerable inconvenience and added cost. An alternative is to apply a temporary but sterile ordinary, non-TNP dressing to the wound and for the patient to be returned to the ward where the non-TNP dressing may be replaced with a TNP dressing and be connected to a vacuum device and, if the dressing leaks it can be replaced or repaired in the ward. This alternative has obvious disadvantages in that there is duplication of effort in providing dressings, added cost in needless dressings and most importantly the risk of infection in exposing a fresh wound to a non-sterile atmosphere.

A further reason why a TNP dressing is ideally applied in an operating theatre immediately after a wound has been debrided is that some times the removal of dead tissue can require cuts close to blood vessels and the subsequent application of a vacuum via a sealed dressing can cause bleeding from blood vessels close to the surface and which may require specific attention. Should this be the case then it is clearly an advantage that this be discovered in an operating theatre having a sterile environment rather than later in a ward if corrective action needs to be taken.

It would clearly be an advantage to be able to test a TNP dressing in a sterile environment without having to sterilise vacuum pumps and the like in order to do so.

It is an object of the present invention to provide apparatus and a method for the testing of TNP dressings in a sterile environment such as an operating theatre, for example, without the need to use conventional vacuum producing apparatus as used in TNP therapy, for example.

According to a first aspect of the present invention there is provided a method for testing the integrity of a dressing applied to a wound for the purpose of applying topical negative pressure therapy to that wound, the method comprising the steps of: applying a dressing to the wound so as to create a sealed cavity over the wound, the cavity having an aspiration conduit operably connected thereto for the purpose of creating a vacuum in the cavity; providing adapter device means to a liquid scalpel so as to create a substantially closed volume around a region in which a water jet exists in operation of the water scalpel; connecting a free end of the aspiration conduit to the closed space formed by the adapter device means on the scalpel; and, using a vacuum created by a venturi effect of the liquid jet in the closed volume to create a vacuum in the dressing cavity.

Although discussed above with reference to the debriding of wounds prior to the application of a TNP dressing the present invention is, of course, relevant to any surgical procedure using a suitable liquid scalpel followed by the application of a TNP dressing.

The water scalpel may be a water scalpel used to effect a debriding operation on the wound in an operating theatre immediately before application of the TNP dressing.

An example of a suitable water scalpel may be found in WO 2003/045259, the content of which is included herein by reference and particularly, though not exclusively, the subject matter relating to FIGS. 12A to 12D thereof.

The water scalpel described in the above reference has a cutting liquid jet at a tip portion thereof, the liquid jet also carries tissue debris into a channel and conduit within a water scalpel handset. In the region through which the liquid jet passes and in particular in the region where the water jet passes into the handset conduit a venturi effect is created with a lowering of pressure in that region. The present invention provides an adapter device and method of using the adapter device to fit on the functional end of a water jet scalpel and form a closed volume around the region in which the venturi effect is created. The adapter device is connected to the cavity formed over the wound by the TNP dressing by means of an aspiration conduit which, if the dressing is adequately sealed will be connected to a vacuum source when the patient is returned to the ward so as to apply TNP therapy to the wound. The vacuum created by the venturi effect of the water jet evacuates the dressing. If the integrity of the dressing is sound the dressing will have a vacuum generated in the wound cavity and which vacuum will persist for a period of time without decaying too quickly. Once the vacuum is established the aspiration conduit can be clamped off and the time taken for it to decay can be measured so that dressing integrity can be assessed. If the vacuum persists the aspiration conduit clamp can be left in place and the patient taken back to the ward. If the dressing integrity is insufficient then the dressing can be repaired or replaced and tested again.

According to a second aspect of the present invention there is provided an adapter device. The adapter device may have a mating portion to receive a liquid scalpel cutting head, the mating portion being dimensioned to suit the particular scalpel proposed for use and which mating portion forms a closed chamber or volume which is susceptible to the pressure drop generated by the venturi effect of the liquid jet. The adapter device further comprises attachment means for affixing the mating portion to the scalpel and aspirant conduit connecting means which is in fluid communication with the closed volume formed between the adapter mating portion and the scalpel.

It is to be noted that the terms “closed chamber”, “closed volume” and “substantially closed volume” used herein mean a volume in which a pressure drop occurs due to the venturi effect of the water jet passing through. The chamber or volume formed by the adapter device is not completely closed since it is necessary for there to be a drainage passage into which the liquid jet is directed and from which it is drained away to suitable waste receptacle or waste processing means and minor leaks can be accommodated. The chamber or volume is, however, closed to the free ingress of ambient atmospheric air.

The adapter device may be made from a flexible or conformable and resilient plastics material to improve the fit and sealing with the liquid jet scalpel.

According to a third aspect of the present invention there is provided apparatus for testing the integrity of a dressing applied to a wound for the purpose of applying topical negative pressure therapy to that wound, the apparatus comprising: a dressing on the wound forming a sealed cavity over the wound, the cavity having an aspiration conduit operably connected thereto for the purpose of creating a vacuum in the cavity; adapter device means attached to a water scalpel, the adapter device and scalpel forming a substantially closed volume around a region in which a water jet exists in operation of the water scalpel; and, a free end of the aspiration conduit connected to the closed space formed by the adapter device means and scalpel.

The apparatus according to the present invention may further be provided with a non-return valve in the aspirant conduit flow path such that after the dressing has been tested for integrity and found to be adequate the liquid scalpel and adapter device may be disconnected from the aspirant conduit without the need for the conduit to be clamped off.

A pressure gauge or pressure indicator of any suitable type may also be attached to the aspirant conduit line so that the level of vacuum being applied to the wound cavity may be measured in order that it does not become too high.

A safety valve may also be included in the aspirant conduit line to prevent the vacuum level in the dressing from exceeding a predetermined maximum.

In order that the present invention may be more fully understood examples will now be described by way of illustration only with respect to the accompanying drawings, of which:

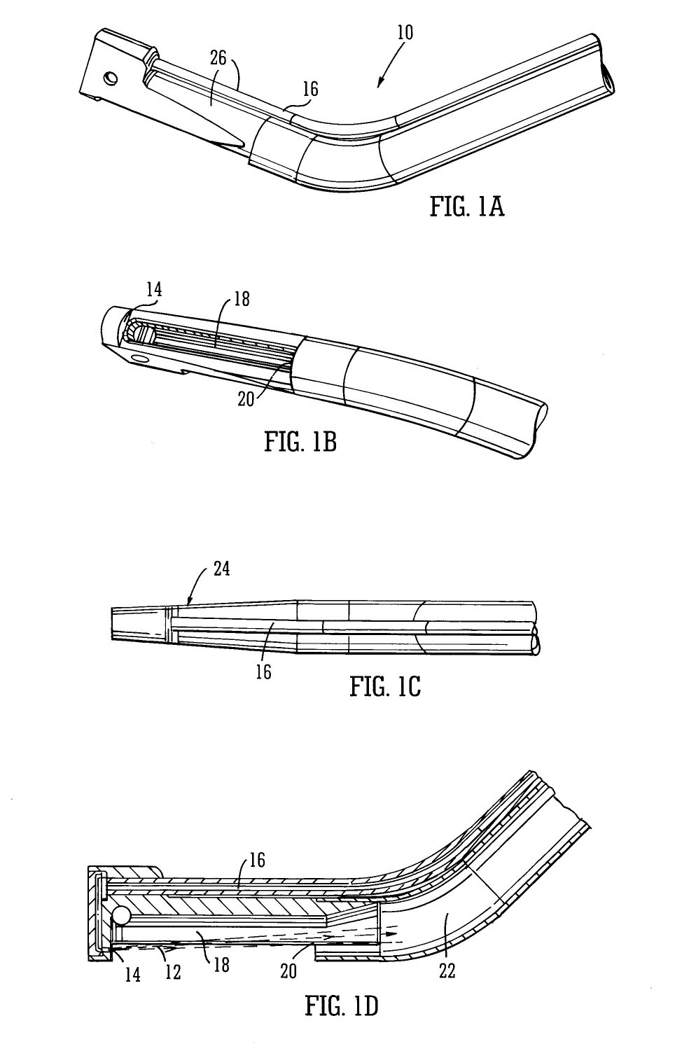

FIG. 1A to 1D show the cutting end of an exemplary liquid scalpel suitable for use in both the method and apparatus and with the adapter device according to the present invention;

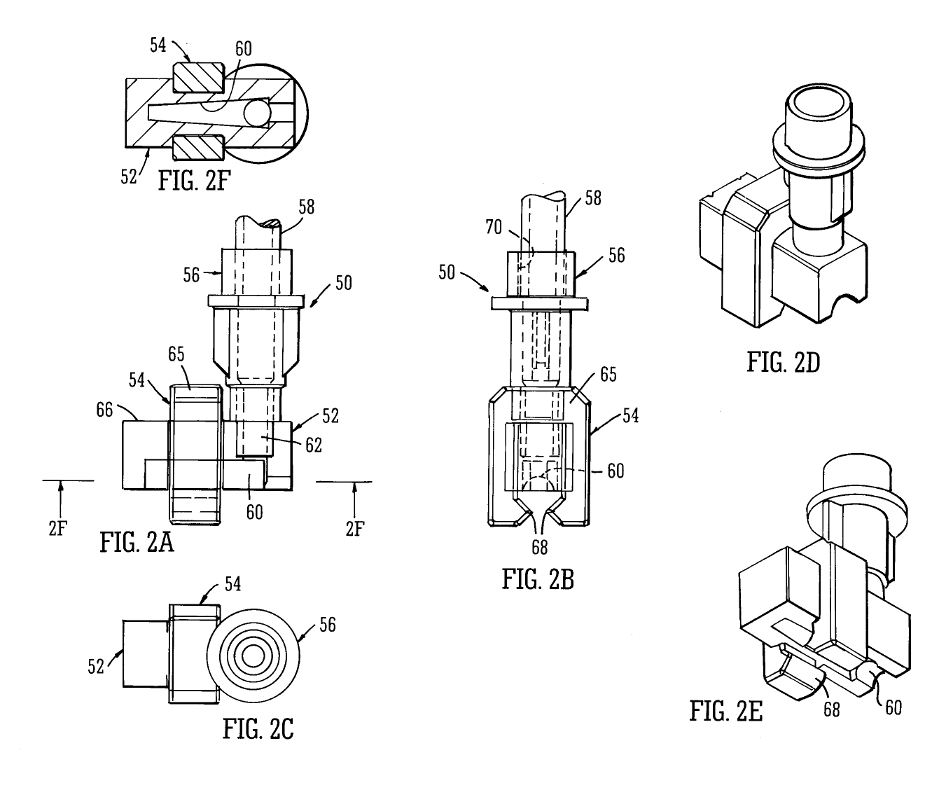

FIGS. 2A to 2F show various views of an adapter device suitable for use with the liquid scalpel shown in FIG. 1;

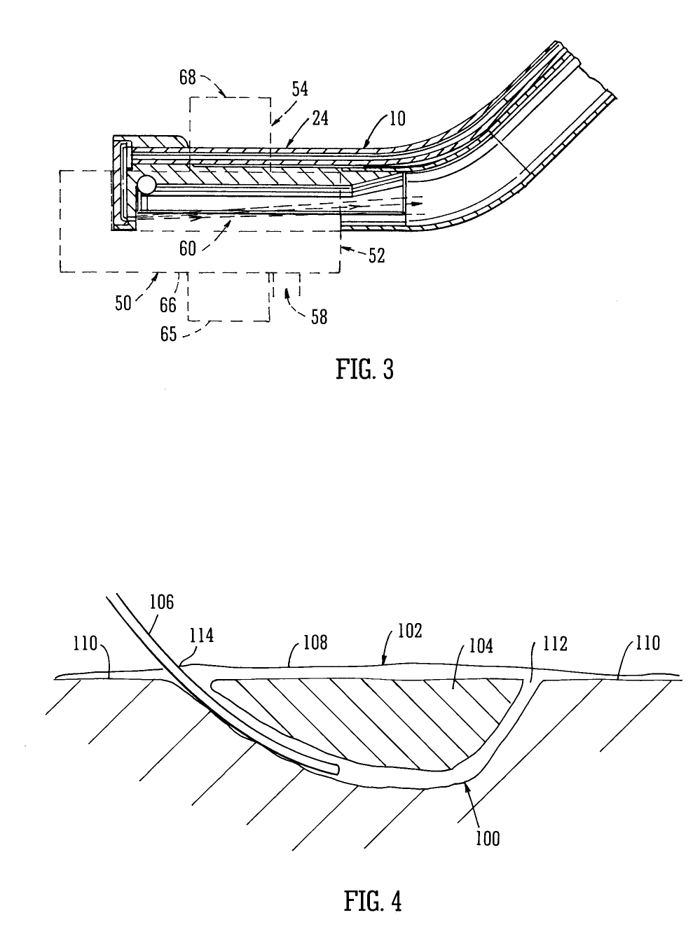

FIG. 3 shows a schematic diagram of the adapter device of FIG. 2 attached to the liquid scalpel of FIG. 1D;

FIG. 4 shows a schematic cross section of a wound having a dressing by which TNP therapy may be applied to the wound;

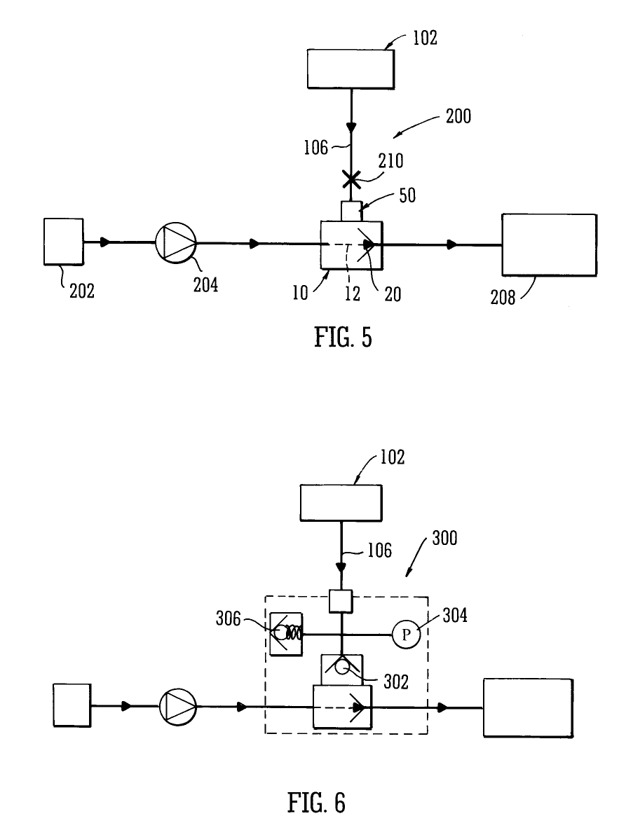

FIG. 5 shows a schematic arrangement of a first embodiment of apparatus according to the present invention for testing the integrity of a TNP dressing; and

FIG. 6 which shows a schematic arrangement of a second embodiment of apparatus according to the present invention for testing the integrity of a TNP dressing.

Referring now to FIGS. 1A to 1D and where: a schematic top perspective view of a working tip of a liquid jet scalpel handset suitable for use in the method and apparatus and with the adapter device according to the present invention is shown at FIG. 1A; a schematic top plan view of the liquid scalpel working tip of FIG. 1A is shown at FIG. 1B; a top plan view of the working tip of FIG. 1A is shown at FIG. 1C; and, a longitudinal cross section of the working tip of the liquid scalpel of FIG. 1A is shown at FIG. 1D.

It is to be noted that FIGS. 1A to 1D may be seen and explained in WO 2003/0452259 at FIGS. 12A to 12D therein together with other information relating to liquid jet cutting scalpels, the content of this document being incorporated herein by reference.

The working tip of a liquid scalpel hand held instrument is shown generally at 10. As is known in the prior art, a liquid scalpel operates by forming a high pressure liquid jet 12 which emerges from a nozzle 14 at a forward end of the instrument, the liquid forming the jet 12 is supplied via a tube system 16 which is supplied by a remote high pressure pump (not shown). The high pressure jet 12 traverses a channel 18 and is directed into an aperture 20 which is at the end of a waste drainage lumen 22 which is connected downstream to a waste receptacle or waste processing container (not shown) to receive jet liquid and tissue debris from the procedure being carried out. The nose 24 of the scalpel has a generally tapered shape in plan view (FIG. 1C) and is of relatively simple geometrical shape. Around the nose 24 are curved side cheeks 26 at a lower portion, remote from the channel 18. In the region of the liquid jet and where it enters the aperture 20 into the waste drainage lumen 22 a venturi effect is established by the flowing jet liquid stream and a pressure drop is created in this region. It is this pressure drop caused by the venturi effect which the present invention seeks to capture.

Referring now to FIGS. 2A to 2F and where: a side elevation view of an adapter device suitable for use with the liquid scalpel of FIG. 1 is shown at FIG. 2A; an end view is shown at FIG. 2B; atop plan view at FIG. 2C; a perspective view from above at FIG. 2D; a perspective view from below at FIG. 2E; and, a cross section through the line 2F-2F of FIG. 2A at FIG. 2F.

An adapter device is shown generally at 50 and comprises a body portion 52; an attachment clip member 54; and a conduit connector 56 for accepting the free end of an aspiration conduit 58 (the remote end of the conduit 58 being operably connected to a TNP dressing as will be explained below). The body portion 52 has a tapered recess 60 therein corresponding to the shape of the nose 24 of scalpel. The body portion further comprises an aperture 62 communicating with the recess 60 and the conduit connector 56. The attachment clip member 54 is of generally “U”-shaped form, the bottom bar 65 of which sits on a top surface 66 of the body portion and inturned fingers 68 grip lower side surface cheeks 26 of the scalpel when the latter is entered into the recess 60. The conduit connector 56 has a socket portion 70 to accept the aspirant conduit 58.

FIG. 3 shows a schematic representation of the adapter device 50 of FIG. 2 attached to the liquid jet cutting scalpel 10 of FIG. 1. The nose 24 of the scalpel is received in the recess 60 in the body portion 52 of the adapter device, the body portion 52 being shown in dashed lines.

FIG. 4 shows a schematic representation of a wound 100 which has been debrided with the liquid jet scalpel 10 of FIG. 1 and has had a dressing 102 applied thereto and of which the integrity needs to be tested prior to a patient being taken from an operating theatre (not shown). The dressing comprises a wound filling material such as a wad of gauze 104, for example; an aspiration conduit 106 which aspirates fluid, both wound exudate liquid and air; and a covering sealing drape 108. The covering sealing drape 108 is well known in the dressing art and has a layer of a pressure sensitive adhesive (not shown) on the face which is adhered to sound skin 110 surrounding the wound. 100. The aspiration conduit 106 is connected during subsequent TNP therapy to a vacuum source such as an electrically powered vacuum pump (not shown), for example, and which creates a vacuum (a pressure below ambient atmospheric pressure) in the wound cavity 112 formed under the dressing drape 108. However, for TNP therapy to be successful it is essential that the drape 108 seals efficiently with the skin 110 surrounding the wound 100 and also with the aspiration conduit which frequently is made to pass through the drape at the point 114 and which drape is pinched around the conduit so as to seal with itself (other means of sealing the conduit 106 may be employed such as grommets and the like and the pinched sealing drape is merely exemplary of one method). Successful TNP therapy is dependent upon the wound cavity being able to maintain a desired vacuum level during operation of the vacuum pump (not shown). Therefore it is necessary to test the integrity of the dressing so as to ensure that there is no gross leakage of ambient air into the dressing.

FIG. 5 shows a schematic representation of a first embodiment of apparatus 200 according to the present invention of testing the integrity of a dressing 102 as exemplified in FIG. 4 to resist unwanted leakage of ambient air into the wound cavity 112 created by the dressing 102. The apparatus comprises a fluid reservoir 202 to supply sterile fluid to a high pressure pump 204 which supplies the liquid jet cutting fluid to the cutting scalpel 10 described with reference to FIG. 1 to create a jet 12 which enters the aperture 20 so as to create a vacuum by means of the venturi effect of the jet 12 entering the aperture 20. Waste cutting fluid is drained away to a waste fluid reservoir 208. The scalpel 10 is fixed to the adapter device 50 described with reference to FIGS. 2 and 3 and which is connected to the conduit 106 of the dressing 102. The vacuum created by the venturi effect applies a vacuum to the wound cavity 112 in the dressing 102 and once a vacuum has bee established in the wound cavity the conduit 106 may be clamped off by a clamp 210 and the dressing observed to determine if the vacuum is being maintained for a sufficient period of time and that decay of the vacuum is not too rapid. If the vacuum appears to be maintained then the clamp may be left in place and the connector 50 may be disconnected from the conduit 106 and the patient removed to the ward where the conduit 106 may be connected to TNP therapy apparatus (not shown).

FIG. 6 shows a schematic representation of a second embodiment of apparatus 300 for testing the integrity of a dressing 102. The apparatus essentially comprises the apparatus features of those described with respect to FIG. 5 but further includes a non-return valve 302, pressure gauge 304 and a safety valve 306. In this embodiment the adapter device 50 may be modified to include apertures/conduits to which the non-return valve 302, safety valve 306 and the pressure gauge 304 may be connected. The non-return valve allows the liquid jet 12 to be stopped and hence the vacuum stopped without having to clamp off the conduit 106 to determine if the vacuum in the wound cavity is being suitably maintained. The safety valve ensures that the vacuum applied to the wound cavity does not exceed a safe maximum and the pressure gauge provides a secondary check to ensure the vacuum level does not exceed a safe level. As with the embodiment described with respect to FIG. 5, the conduit 106 may be finally clamped off if the vacuum is adequately maintained and the patient returned to the ward as before.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. A method for testing the integrity of a dressing applied to a wound for the purpose of applying topical negative pressure therapy to that wound, the method comprising:
applying a dressing to the wound so as to create a sealed cavity over the wound, the cavity having an aspiration conduit operably connected thereto for the purpose of creating a vacuum in the cavity;
providing adapter device means to a liquid scalpel so as to create a substantially closed volume around a region in which a water jet exists in operation of the water scalpel;
connecting a free end of the aspiration conduit to the closed volume formed by the adapter device means on the scalpel; and
using a vacuum created by a venturi effect of the liquid jet in the closed volume to create a vacuum in the dressing cavity.

2. A method according to claim 1, further comprising providing a non-return valve between the dressing and the liquid scalpel.

3. A method according to claim 1, further comprising monitoring a time for a vacuum in the dressing to decay.

4. A method according to claim 1, further comprising providing pressure monitoring means.

5. A method according to claim 1, further comprising providing safety valve means to limiting a maximum vacuum applied to the dressing.

6. An adapter device for use in the method of claim 1.

7. An apparatus for testing the integrity of a dressing applied to a wound for the purpose of applying topical negative pressure therapy to that wound, the apparatus comprising: a dressing on the wound forming a sealed cavity over the wound, the cavity having an aspiration conduit operably connected thereto for the purpose of creating a vacuum in the cavity; adapter device means attached to a water scalpel, the adapter device and scalpel forming a substantially closed volume around a region in which a water jet exists in operation of the water scalpel; and, a free end of the aspiration conduit connected to the closed space formed by the adapter device means and scalpel.

8. An apparatus according to claim 7, further including a non-return valve in the aspirant conduit flow path.

9. An apparatus according to claim 7, further including pressure monitoring means.

10. An apparatus according to claim 7, further including a safety valve in the aspirant conduit line.

11. An adapter device for the apparatus of claim 7, wherein the adapter device comprises:
a mating portion to receive a liquid jet scalpel cutting head, the mating portion forming a closed chamber or volume which is susceptible to a pressure drop generated by a venturi effect of the liquid jet;
attachment means for affixing the mating portion to the scalpel; and
aspirant conduit connecting means which is in fluid communication with the closed volume formed between the adapter mating portion and the scalpel.

12. An adapter device according to claim 11, wherein the device is made from a flexible, resilient plastics material.

13. (canceled)

14. (canceled)

15. (canceled)
